(19) United States
(12) Patent Application Publication
(10) Pub. No.: US 2007/0213617 A1
(43) Pub. Date: Sep. 13, 2007

(54) Infrared detection of cancerous tumors and other subsurface anomalies in the human breast and in other body parts

(76) Inventors: Herbert L. Berman, Los Altos Hills, CA (US); Carol A. Tosaya, Los Altos, CA (US); John W. Sliwa JR., Los Altos, CA (US)

Correspondence Address: David W. Collins;Intellectual Property Law, Suite 100, 512 E. Whitehouse Canyon Road, Green Valley, AZ, 85614, US

(21) Appl. No.: 11/706,120

(22) Filed: Feb. 14, 2007

Related U.S. Application Data
(60) Provisional application No. 60/774,562, filed on Feb. 16, 2006.

(51) Int. Cl. A61B 6/00

(52) U.S. Cl. 600/473; 600/474

(57) ABSTRACT

Apparatus and methods to further improve the performance of breast IR-imaging are provided, employing a combination of near-IR and mid-IR frequencies for detection of cancer and other types of subsurface defects. In addition, an IR transmissive or transparent window that can be IR-imaged through is disclosed that may also be utilized to one or both of distort the breast and/or manipulate an artificial heat-flow into or out of the breast.

### CROSS-REFERENCE TO RELATED APPLICATION

The present application claims priority from provisional application Ser. No. 60/774,562, filed Feb. 16, 2006.

### BACKGROUND OF THE INVENTION

1. Field of the Invention

The present invention is directed to the improved detection of cancerous tumors and other subsurface anomalies in human organs or body parts and, in particular, to cancerous growths in the human breast, and, even more particularly, to the use of infrared optical wavelengths for such detection.

2. Description of Related Art

It has been known from mid-IR thermal-imaging or “thermography” that subsurface breast-cancer tumors present observable infrared thermal (mid-IR) contrast on the external breast tissue surface; however, that contrast is substantially hidden among surface thermal mid-IR contrast or clutter caused by non-anomalous breast vasculature situated at the surface and/or at-depth and having its own thermal mid-IR surface signature. This results in thermal-IR images of breasts that are difficult to interpret correctly, manually or with computer help, in terms of locating the warmer anomalous tumors with certainty.

It is known, for example, that precancerous and cancerous breast tissue portions typically generate more heat than normal healthy tissue portions and result in nearby hot spots on the skin as hot as 2.5° C. above their immediate surroundings, depending on their size and depth. Using thermal infrared or “thermal-IR” thermography imaging, generally done in the 8-14 micron wavelength regime, it has been demonstrated by several groups over several decades that such underlying tumors have a tissue-surface thermal-IR “heat” signature, albeit that signature is notoriously noisy and currently not alone sufficient to accurately identify such cancers or pre-cancers. What is certain is that prior art thermal-IR imaging is sufficiently sensitive to not only see tumors but to also see pre-cancerous tissues if they are not masked by such confounding thermal mid-IR contrast. This is not surprising, given modern thermography's sensitivity of better than 0.1° C. However, these thermal images have been noisy in nature and subject to many physiological and environmental factors such that their diagnostic accuracy, in terms of false positives and false negatives, needs improvement. Environmental factors that are known to contribute to noise also include variations in room temperature and room air circulation, As mentioned, underlying vasculature that is frequently close to the tissue surface also presents substantial confounding mid-IR thermographic contrast on the tissue surface.

Prior investigators have attempted to selectively enhance the underlying tumor's thermal mid-IR signature, which is viewable only on the breast-tissue surface because mid-IR wavelengths do not appreciably penetrate tissue. One such enhancement involves employing what is called thermal stress imaging. In thermal stress imaging, one looks at a breast which has been physically cooled and/or has been vasoconstricted.

A physically surface-cooled breast shows enhanced thermal contrast from any embedded heat-producer upon rewarming; unfortunately, these embedded heat-producers also usually include veins and arteries. Physical cooling of the breast may or may not provide vasoconstricting action.

Vasoconstriction, however, is a nervous system driven closure of the veins as caused by dipping the feet or hands in ice water. That is, all the veins in the body will vasoconstrict for as much as 15 minutes from such a short exposure, even if the breast itself is not exposed. This works for 80% plus of patients but unfortunately not for all patients. Note that vasoconstriction beneficially would reduce the thermal signature of the veins in the breast without requiring direct cooling of the breast itself. Typically, using physical cooling and/or vasoconstriction, one thereafter thermographically observes the re-warming of the breast. This is the current state of the art wherein vasoconstriction and/or physical cooling is employed to somewhat enhance contrast.

In any event, these two thermal stress imaging measures, whether used alone or together, marginally improve thermal contrast. In some patients, vasoconstriction is not reliable and cannot be used at all

Prior art breast infrared imaging of the last couple of decades or so has utilized mid-IR wavelengths, which is generally defined as a wavelength or wavelength window containing the 8 to 14 micron wavelength range at which thermal-IR energy is at its peak output from human tissues. This wavelength emanates only from the surface top few microns of thickness and therefore represents only surface hotspots (or coldspots) of underlying heat-producing (or heat-sinking) features. Thus, this is not looking under the surface in a direct way. Such wavelengths cannot penetrate tissue appreciably, so when one observes hotspots on the tissue surface using mid-IR, one is seeing only that heat that has conducted to the surface from the tumor underneath. So it is an indirect imaging technique for subsurface heat-producers.

Unlike mid-IR thermal energy, shorter near-IR non-thermal energy can emanate from tissue features at depth in a somewhat unhindered direct manner, despite considerable scattering and moderate attenuation. In other words, these shorter or near-infrared (NIR) infrared waves are substantially more penetrating in tissue. Thus, investigators today are developing multi-purpose contrast agents that are directly visible at-depth in the near-IR in order to selectively visualize subsurface contrast-decorated features such as cancer. Typically, such near-IR contrast agents are excited into near-IR emission by a separate excitation radiation of an optical or electromagnetic nature. The near-IR, as defined below, comprises IR wavelengths in the 1-3 micron regime and below. Within this range, there are well known transparent windows in tissue for near-IR as well as a few specific wavelengths whereat hemoglobin and other molecules selectively interact with the near-IR radiation with predictable attenuation or lack thereof.

The reader should note then that a tumor, using near-IR (NIR) and mid-IR (MIR) imaging techniques, would show a tissue-surface mid-IR thermal hotspot and show an at-depth NIR contrast, particularly if decorated with a cancer-finding NIR contrast agent. The co-location of these two wavelength types of contrast further assures that a cancerous tumor is being seen. This is because the NIR emission or absorption contrast-agent characteristics will only be at tumors that have been selectively decorated. Therefore, a key attribute of the invention is the option of using both MIR and NIR to sort out which features are tumors and which are healthy tissue and/or vasculature. Note that the invention does not require the use of a NIR contrast agent, as subsurface features and blood have some inherent NIR contrast as well.

So it will be appreciated that the prior art breast thermal-imaging or thermography technology, which has been commercially fielded and is still being sold, images breast surface tissue only using mid-IR wavelengths and is really looking only at surface hotspots and coldspots. So essentially what one sees is all manner of hot-spots and thermal blooming as caused by near-surface and subsurface tumors and/or vasculature and/or perfusion variations as a function of capillary structure and tissue type. Breast cooling and vasoconstriction with subsequent re-warming and/or removal of vasoconstricting influence help, but not a lot. The bottom line is that thermography still is not as widely used nor as trusted as is its competitor, the not-hugely better mammography modality. At this time, the FDA has long-ago approved thermography as an adjunctive to mammography, but thermography does not hold an authoritative clinical position in terms of wide acceptance and reimbursement.

The invention here offers advancements in two areas, both of which can help thermography. The first is improvements to the mid-IR thermography technique itself and the second is near-IR or NIR plus MIR imaging wherein both data or image types are co-analyzed to sort tumors from normal tissues and vasculature.

Prior art breast thermal imaging or mid-IR thermography has typically involved taking a few noncontact breast images from various viewing angles before and after vasoconstriction and/or alcohol spraying or blowing cool air (cooling), for example. Sprayed alcohol and blown cold air still allow for real-time transient imaging. Unfortunately, blown air has poor and non-uniform heat transfer abilities on a three-dimensional breast and sprayed evaporating alcohol is also non-uniform as well as unpleasant if not harmful to skin and respiration. All such forced physical cooling measures have large variations related to breast shape and orientation.

Despite these prior art incremental improvement measures, the current state of affairs is still that the FDA has approved prior art breast-cancer thermal or mid-IR imaging or thermography only as an adjunct diagnostic method, meaning that it can be used only to provide supplemental information beyond that provided by another diagnostic technique such as mammography and/or clinician palpation. Given the still-problematic variable and marginal signal-to-noise ratio of the existing thermal-IR imaging or thermography prior art, this is quite understandable and correct. However, it would be of substantial benefit to improve IR imaging of the breast such that IR-imaging would instead become either a superior standalone diagnostic or a stronger adjunct or equal to mammography. Mammography itself is far from perfect and probably cannot itself be improved much more beyond making it tomographic rather than 2-D in nature. Essentially, mammography is the current “gold standard” because it is the perceived best of several non-optimal technologies.

### SUMMARY OF THE INVENTION

The various embodiments of the present invention are directed to infrared or IR detection of breast cancer tumors and other subsurface anomalies in the human breast.

In a first embodiment, apparatus for such detection comprises a means for gathering mid-IR (MIR) image data of the surface heat pattern of the tissue from at least one angle noninvasively or invasively. The apparatus further comprises a means for gathering near-IR (NIR) image data of at least some subsurface structure of the tissue from at least one angle noninvasively or invasively. The apparatus also comprises a means for correlating at least some subsurface data with at least some surface data in a manner wherein the location, size or risk of a heat-producing anomaly can be ascertained with improved certainty over that achieved using mid-IR wavelengths alone.

In a second embodiment, the IR imaging apparatus incorporates a means for manipulating, physically or thermally, the underlying tissue structures utilizing an IR (mid-IR and/or near-IR) transparent window through which at least some of the imaging is performed. Because the “window” is placed against the skin, it can beneficially be used to squeeze or shear the skin or breast tissue as a whole. Squeezing has several benefits including (a) vasculature can be squeezed shut, thereby limiting its thermal contribution, (b) a large area of tissue under investigation is brought in a flat normal incidence angle with the imaging device aiding measurements and removing the angular dependence of emissivity, and (c) if a liquid or gel optical couplant is employed as a thin film between the window and the breast, it can serve to control breast tissue emissivity and emissivity uniformity as well as assure good optical contact. Shearing via rotational or sliding motion of the window drags the surface tissue but “leaves behind” underlying tissue due to tissue shear deformation. Deeper tissues shear less than surface tissues. Thus, by taking images (MIR and/or NIR) at two such sheared positions, one can deduce the depth of the tumor because its surface hotspot moves during the shearing process an amount proportional to its depth.

In a third embodiment, a preferably IR (mid-IR and/or near-IR) transparent window is utilized as a means of delivering or removing heat from the breast. The window adds or removes heat from the breast either because of its own heat capacity and conductivity or because it includes or is used with a heating or cooling means such as a flowed coolant, electric heater or radiant heater. In any event, heat can be injected or removed in a much more controlled manner and at a much faster and more uniform rate than using prior art cooling means such as blown air or sprayed alcohol. The heat-manipulating window may also be used to thermally cause vasoconstriction in susceptible patients. It may also be used to maintain a large thermal gradient versus tissue depth, thereby further enhancing the thermal contrast of deep tumors. This “heat-plate” or “cold-plate” variation does not require use of an IR transparent window, as it could be used and removed for subsequent imaging. However, we prefer it to be IR transparent and left in place during imaging.

Included in the scope of the invention is the integrated use of another imaging modality with any of the above embodiments. For example, one could use two of our IR windows and squeeze a breast between them. Those familiar with mammography will realize that this arrangement is physically similar to a mammography machine. The same applied to ultrasound imaging. There has been some research seen in the industry involving a mammography machine whose squeezing-plates also act as the face(s) of acoustic imaging transducers.

### BRIEF DESCRIPTION OF THE DRAWINGS

FIG. 1 depicts a sectional view of a human breast containing subsurface tumors, near-surface tumors as well as near-surface and subsurface vasculature, wherein an embodiment of the inventive infrared imaging apparatus is depicted imaging the breast while an external heat-flow is introduced along with an external deformation force.

FIGS. 2A-2B depict a sectional view (FIG. 2B) and a corresponding top plan view (FIG. 2A) of a breast tissue region wherein a tumor generally underlies an overlying vasculature portion and both NIR and MIR wavelength image data is analyzed to better identify the underlying tumor versus the prior art consideration of only MIR thermographic data.

FIG. 2C depicts a plot of IR contrast along the lumen shown in FIG. 2A with just MIR and with MIR as corrected by consideration of the NIR signal.

FIG. 2D depicts what the combined MIR and NIR-corrected image might look like at the tumor and overlying vasculature of FIG. 2A.

FIGS. 3A-3B depict similar tissue views as FIG. 2B, where the tissue is depicted in uncompressed and compressed states wherein the compression is applied with an IR-transmissive or transparent plate or window, in accordance with the second embodiment of the invention. The windows of FIGS. 3A-3B are also shown acting as tissue heat-removal means as described in the third embodiment.

### DETAILED DESCRIPTION OF THE INVENTION

The present inventors herein provide new apparatus and methods to further improve the performance of breast IR-imaging, and indeed of any IR-imaging technique used to image any type of subsurface defect in any type of target anatomy or object. It will be seen that the invention is particularly, but not exclusively, applicable to deformable objects to be inspected for subsurface defects or anomalies which result in thermal-IR defect-signature components at the object surface. Inspection of objects such as the human breast can utilize all three embodiments of the invention or just one of the embodiments of the invention. The first embodiment, that of utilizing combined MIR and NIR wavelengths, can be used so long as the imaging means support both wavelengths, not necessarily simultaneously. The second embodiment, the preferably IR transparent window with which tissue is squeezed or sheared and which provides for uniform emissivity, can be used for just MIR thermography provided it is MIR transparent or with NIR subsurface imaging provided it is NIR transparent or with both assuming transparency to both. The third embodiment, that wherein a cold plate window is utilized, requires that the window be capable of delivering or removing heat from tissues with the help of its specific heat capacity and/or heat manipulation means coupled to it. Note that the cold plate window will likely squeeze the breast for good thermal contact. Such contact may or may not also be used to utilize the second embodiment.

In the first inventive embodiment, multiwavelength (MIR plus NIR) infrared detection or imaging is utilized to overcome the above-discussed problems of vasculature being confused with tumors in MIR images. We stress that the MIR and NIR images may be taken or sampled simultaneously or sequentially by one or more image sensors. Many CCD and CMOS imaging chips in digital cameras can image both in the visible and in the NIR and we include the use of such devices to collect visible and NIR overlaid or combination images. Typically, the MIR thermographic sensor may be a dedicated image sensor or camera.

Because mid-IR imaging superficially “sees” only surface-evident thermal signatures (hotspots) of underling features but near-IR spatially “sees” non-thermal direct optical contrast of surface and subsurface features emanating “through the skin” from various depths, then there are really two different ways of looking at the same features: one indirect and superficial (thermal-IR) and one direct and through the tissue depth (near-IR). The present inventors realized that, because of the fundamental difference between these two image contrast components and their origins, they could manipulate the taking and/or processing of two such images in a manner to separate out which features are due to breast tumors and which are due to normal tissues such as near-surface vasculature. The present inventors realized that, for example, thermal-IR images are “bloomed out” in that their contrast includes the effects of heat spreading in two and three dimensions as the heat makes its way to the surface. Near-IR non-thermal image contrast is limited to the dimensions of the structure being imaged. Thus, one can immediately, by image comparison methods, for example, determine along a line of sight through an apparent surface hot spot where the actual physical tumor seen in NIR is located under the hotspot seen in MIR. Further, the inventors recognized that because the NIR sees directly to at-depth objects, then by looking at more than one perspective angle or line of sight, one can estimate the physical depth of tumors beneath their surface-evident hot spots. Further, knowing the size of the tumor from it multiple-perspective derived depth, one can compare it to its surface hotspot and deduce or calculate how hot the actual at-depth tumor is to cause that hot spot thereby giving a quantitative clue as to its seriousness. Applicable also to the present invention herein any any of its embodiments is the use of known NIR fluorophors or contrast agents, which are administered to patients and which can “decorate” features such as subsurface blood-filled features, including, but not limited to, tumors or vasculature. Such contrast agents further improve the optical contrast of the features in the near-IR as observed from the tissue surface, preferably while utilizing an optical excitation source that causes the generation of near-IR at the selectively contrast-decorated features.

So given the availability of NIR and MIR imaging, we can utilize such image data in several manners to sort out what are tumors and what is vasculature. For example, from a single viewing position, one will see the surface MIR contrast of tumors and vasculature but will also see underneath that the NIR contrast of the actual tumors and blood-filled lumens or vasculature. Because of thermal blooming effects, the hot spots will be laterally larger in the MIR than the actual anatomical features as seen in the NIR. The amount of blooming will be proportional to feature depth.

Also, given both types of wavelength data or images, one can utilize two or more viewing angles or perspectives which will show, for example, a subsurface tumor or blood vessel in the NIR moving relative to its overlying MIR hot spot. This apparent motion between the image types can be used to compute the physical depth of the tumor or lumen. One also knows the actual sizes of the features with two or more views thus can perform heat flow calculations and deduce intrinsic heat output.

It will be realized that tumors and vasculature will generally show both MIR and NIR contrast. A skin infection might show only MIR contrast and the lack of the subsurface NIR image would indicate that that hot spot is indeed either a skin infection or, more rarely in the breast, a skin cancer. Likewise, an object seen at-depth in the NIR but not seen in surface MIR would not likely be a heat-producing tumor. The relative amounts of NIR and MIR contrast can be substantially independently manipulated as by utilizing the second and third embodiments discussed below to help sort out details of the actual tumors, if any. Included in our inventive scope is any manipulation of such images or image data in a manner coaxing out the needed information regarding the tumors or lack thereof. These would include techniques such as image subtraction, multiplication, border and edge-finding, gamma curve adjustment, feature recognition and spatial transformations, particularly for multi-axis views. The important point here for the first embodiment is that the user now has two independent types of data as well as direct access to the depth dimension using oblique or angled viewing.

Further, using existing physical breast cooling techniques and observing the re-warming of the breast we will see the thermal blooming reduced by cooling and then gradually increased again but still centered upon the NIR contrast of the underlying heat-producing object. The cooling typically still increases the contrast relative to healthy tissue background especially during the re-warming period.

Using prior art vasoconstriction via cooling of appendages other than the breast we will likewise see the thermal blooming of vasculature reduced upon cooling and centered over the underlying NIR contrast of the lumens themselves. Vasoconstriction will improve somewhat the MIR contrast of tumors as the MIR contrast of vasculature is reduced by vascular constriction. The fact that a feature's thermal MIR signature is reduced by vasoconstriction but the features NIR image contrast doesn't change as much is an excellent indication that what you are looking at is a vein with reduced blood flow. This is because the heat output is related to the vein diameter squared whereas the apparent NIR size is related to the diameter directly.

In a second embodiment, an IR-transparent window (MIR and/or NIR) is utilized through which IR (MIR or NIR) imaging is performed and which may be utilized as a tissue-manipulation or deformation means and/or a means to control tissue emissivity. The inventors realized that an IR contact plate or window that grips the tissue against sliding can be used to deform the surface tissues relative to the deeper tissues. By looking through the window in the NIR and/or MIR, we would expect to see several revealing phenomena. The first is that any heat-producing feature below the surface will shift position relative to its former surface hot spot. That is to say, if the window is twisted or translated, the old hot spot will, over a thermal equilibration time, move to the new overlying position. A second phenomenon is that the plate or window can be used to squeeze the tissue. Such squeezing can be utilized to reduce vasculature blood flow or arterial blood flow, thereby reducing the vasculature MIR signal with or without added vasoconstriction. Typically, it will be surface or superficial lumens that will be most squeezed relative to deeper ones. This helps greatly in suppressing surface vasculature MIR contrast. A third phenomenon is that the optical emissivity of the skin can be better controlled using the window plate because one can place a liquid or gel couplant in the plate/tissue interface having desired optical properties and/or one can deposit desirable antireflection or filtering films on the plate. Also, during compression, a large area of the breast is assured to be normal to the observing MIR or NIR sensor. A fourth phenomenon is that the distances between the underlying features and the overlying tissue surface and window can be reduced by such compression or twisting. This brings the features closer to the surface and can also alter even their NIR contrast as their apparent squeezed dimensions change and the blood flowing through them changes in amount and/or gas composition. Note that in instances wherein blood hemoglobin peaks, for example, are interfering, one can substantially remove or squeeze out much of the blood by such squeezing.

Moving now to the third embodiment, we have a plate or window that is utilized to cool, heat or otherwise control the temperature of a tissue portion. For this embodiment, if the plate is an IR transparent window (MIR and/or NIR), then inventive IR imaging can take place through the plate or window while it also performs the thermal manipulation of this third embodiment. Such simultaneous imaging might include the first and/or second embodiments. The third embodiment does not absolutely require that the contacting plate be an IR window, as one could place the plate on the tissue for a limited period, remove it, and IR image the transient effects. However, the present inventors prefer that the plate be an IR window such that it can stay in place to do IR imaging through while allowing simultaneous temperature control or manipulation.

There are several ways to utilize a plate or IR window to heat or cool tissue or to temperature control tissue on which it is laid or held in very close proximity. We prefer physical contact, but include proximity placement to tissue wherein any gap is air filled or is filled with a liquid or gel, which may be static or flowed. The first way to transport heat to/from the breast is to pre-cool or pre-warm the plate or to otherwise assure a plate starting temperature. To do this, one could simply place the plate in a temperature-controlled environment for a period of time and then remove it and place it on the breast. A second way to do this is to incorporate a heater or cooler means in the plate itself or in a proximity gap between the plate and the tissue. As an example, the plate may incorporate thin-film heaters or thermo-junction cooling devices as well as some thin-film or discrete thermistors. For cooling of the breast using such a plate or window, it is desirable not to condense water vapor in the optical path, so we expressly include in the scope of the invention the use of thermal insulation and or dry gas in, on or around the plate to prevent this.

The plate could also incorporate IR transparent flowed liquid coolant that does or does not physically pass through the imaging field of view. If it does not pass through the field of view, it does not necessarily need to be IR transparent.

We include in the scope of the invention plates or windows that are not flat and plates or windows which are not rigid. For example, a shaped-molded cup-shaped plate could be made of mid-IR transparent polymer as is used in home-security cameras. The plate/window may incorporate other optical coatings and may be fabricated of a material that has beneficial thermal properties depending on any thermal function delivered. For example, the plate may have a high or low thermal conductivity or specific heat. The plate may comprise one or more layers or components, some of which act as thermal conductors and others as thermal insulators. The plate may be held to the tissue as by using force, an adhesive or even by vacuum or strap/belt features. The plate may reduce MIR vasculature emission via squeeze-down of vasculature blood flow; however, that mechanism does not exclude the possibility of the alternative or additional use of prior art vasoconstrictive approaches. The reader will likely be aware that vasoconstriction is selective to healthy tissue and does not happen in tumors, thus, its ability to enhance tumors.

In any of the three embodiments, one may utilize NIR and/or MIR optical energy of a passive or active nature. By this we mean, for example, that for NIR, one might deliver NIR radiation to or through the tissues to help enhance NIR contrast. In a different approach, one might deliver infrared MIR to tissues as with an IR lamp, using it to perform pre-warming or re-warming. Thus, one might image natural out-coming MIR or NIR or might image reflected in-going MIR or NIR, or do both.

The inventive IR window may have any shape or pliability including flat, cup-shaped, rigid or flexible and may even be custom-molded to the patient. By applying force to the breast with the inventive IR window, then any of compression, tension, suction or shear can be delivered to the tissues.

We explicitly note that although we use the human breast as our demonstrative example, the present invention is applicable, generally, to the identification of tumors and other MIR and/or NIR abnormalities in various other organs such as the brain, liver, kidney, etc. Finally, the invention is also generally applicable to the spatial location of features in a test object, even in cases wherein none of the features produce anomalous heat and can only be seen in NIR. Thus the invention, particularly the second embodiment, doesn't necessarily require the use of MIR wavelengths.

Finally, the inventive IR and/or cooling window also serves to virtually eliminate variations caused by prior art environmental factors such as room temperature variations, room drafts and breast shapes interacting directionally anisotropically with flowed air. Note that the inventive plate can be made and operated to have very reproducable heat removal or injection despite who is using it. Given the heat transfer capability of a plate the room temperature and breezes can be ignored. The amount and rate of heat transport can also easily be much larger and more prolonged given the solid/liquid nature of the window versus blown air. The present inventors also anticipate that the large cooling capacity of our thermal plate or window will allow for direct thermal-driven breast vasoconstriction to a larger degree than blown air or sprayed alcohol ever could.

Discussion of the Figures

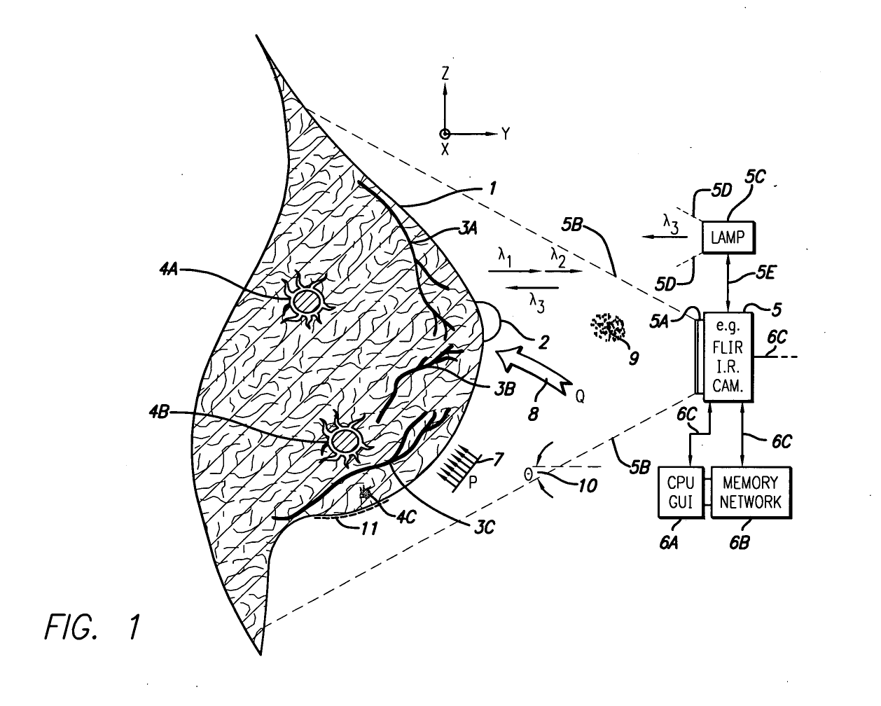

Turning now to the Figures to explain the invention in detail, we see in FIG. 1 a female human breast 1 in sectional view. The breast projects rightward and has a nipple 2. For convenience, we have shown a coordinate system at the top of FIG. 1 with Y- and Z-axes in the plane of the figure and the X-axis emanating from the paper toward the reader.

Within the anatomical structure of breast 1, we see surface and near-surface vasculature or blood-lumens 3A, 3B, and 3C. These are typical normal blood vessels located at least just under the tissue surfaces and/or somewhat deeper. Veins are frequently closest to the surface, smaller and largest in number. Arteries are frequently fewer, larger and deeper. Blood vessels vary in size and location from patient to patient and vary even between the two breasts of a given patient. In any event, this vasculature or lumen-set 3A, 3B, 3C, in the prior art thermographic mid-IR or MIR heat exam, would offer its own IR-heat or thermographic signature component at the tissue surface due to the flow of warm blood in the vessels, causing heat to be deposited at the surrounding and overlying tissue such that surface heat patterns are created. Also shown in FIG. 1 are three subsurface cancerous tumors 4A, 4B, and 4C. It will be noted that tumor 4A is quite deeply situated, whereas tumor 4B is nearer the breast surface. Tumor 4C is just under the tissue surface. All three of the tumors 4A, 4B, 4C are situated substantially behind or over the venous and arterial lumens (hereafter collectively called vasculature) 3A, 3B, 3C when observed from the right or from the +Y region. What that means is that all thermal MIR signatures of tumors 4A, 4B, 4C or lumens 3A, 3B, 3C will all be seen at the breast surface in an overlapped manner. In other words, looking leftwards from the right with a prior art thermal IR (MIR) camera, the thermal MIR signatures of tumors 4B and 4C will overlay the thermal MIR signature of lumen 3C, for example. The MIR intensity or temperature reading of each will be related both to how hot the subsurface feature is and how deep or far away from the tissue surface it is.

On the right hand side of FIG. 1 we see an inventive IR-imaging means 5, preferably also having an infrared lens 5A looking leftward at breast 1 with a cone-shaped or rectangular field-of-view generally defined by phantom lines such as lines 5B. It will be noted that the inventive IR camera or imaging device 5/5A includes or is communicative with a CPU or calculation means and a GUI or graphical user interface situated in schematic function block 6A as well as with some memory and a data bus shown situated in functional block 6B. Further, we depict an illuminator or exciter 5C, typically used herein to illuminate with or excite near-IR radiation. Preferably, the indicated electronics and user interface are provided in the form of a standalone or embedded personal computer or workstation along with the IR camera or imager body 5/5A. Data, power and signal lines 6C are indicated to tie these functions together. A network connection is also depicted passing rightwards off the page. Connection lines 6C may, some or all, be in the form of hardwired or wireless electronic or optical connections or radiowave-type connections such as wireless access point connections to a network. Preferably, modules or components 6A and 6B, if not also 5/5A, are housed in a single box or cabinet.

From previous discussion of our three embodiments, it should be clear that many implementations thereof may utilize both MIR and NIR wavelengths; however, the second and third embodiments can also be practiced using only MIR or only NIR.

Note in FIG. 1 that we depict IR wavelengths or energies passing rightwards and leftwards. As an example, λ₁ and λ₂ traveling rightward might be or contain mid-IR and near-IR wavelengths, respectively, whereas λ₃ traveling leftwards might be or contain near-IR illumination and/or illumination that excites resulting rightward near-IR waves λ₂. Thus, λ₃, which is shown emanating from light source 5C, might be of a pulsed or continuous intensity and might excite or deliver NIR waves in the tissue, such as at an excitable NIR contrast agent or such as by purely reflective NIR contrast mechanisms. In another embodiment, λ₃ is intense mid-IR used to transiently heat the tissue surface or to heat our plate or window.

The reader will note that the IR viewing-angle of the breast 1 in FIG. 1 is variable, both because the breast has a curved shape and because of the angular limitations of the field of view as depicted by lines 5B, which are at an angle θ to the camera central axis and indicated by θ angle 10. In other words, the angle between the breast tissue surface and the IR camera image plane is variable within the field of view. This results in, from point of view of the IR camera 5, an apparent IR emissivity variation across the field of view. Note also that any illuminator 5C may also have a limited beam as depicted by phantom lines 5D. Typically, if an illuminator 5C is used herein, it will have a signal and/or power connection 5E so that it can be activated at appropriate times. Any illuminator 5C would have a controlled illumination angle(s) relative to the breast and/or IR camera 5. The illuminator may be operated in a pulsed or constant mode and would likely be controlled by the CPU 6A. An illuminator 5C might also comprise a scanned beam or point ingoing to the breast. By the same token, the IR camera is preferably a 2-D sensing array, but may also be a point-scanned or rastered device.

We have also indicated in FIG. 1 a thermal heat-flow Q designated as item 8. This heat flow 8, like the mentioned prior art breast cooling flow of heat, is typically practiced to attempt to enhance the surface-visible IR or MIR-heat contrast components of the tumor 4A, 4b at the expense of the vasculature 3A, 3B, 3C IR-heat components. Additionally, we have shown in FIG. 1 a tissue surface pressure or force P designated as item 7 as well as a breast tissue coating item 11. P can optionally be inwardly, outwardly or laterally directed relative to breast 1 and might be dynamic or static in nature. The heat flow Q may be either the prior art thermal stimulation methods or may be our inventive plate/window thermal manipulations. The pressure P, for example, may be applied by our inventive plate/window which may or may not cause the tissue to become flat and face the camera 5, depending on the plate's shape. In this first Figure, we show only the schematically-depicted force P and not the inventive plate itself, if used, which is shown in the later Figures. Schematically depicted coating 11 can be, for example, a coating or contact liquid/gel associated with the inventive plate and designed to minimize undesired optical reflections or to help control emissivity of one or more optical interfaces or surfaces.

All objects emit thermal mid-IR or MIR radiation from their surfaces if they are above absolute zero (all real objects indeed are) and the total radiation energy emitted is proportional to the emitting surface area and to the fourth power of the absolute thermal temperature of the emitting surface. Human skin or tissue is very emissive in the thermal-IR or MIR and this property can easily be utilized to thermographically measure the tissue surface temperature with respectable accuracy of a tenth of a degree C. and at a 2-D frame rate of 10-30 or more frames per second such that thermal transients can be recorded or observed with 1 mm lateral resolution or better.

Thermography, or mid-IR (MIR) infrared imaging, has been known to be able to detect or image infrared surface-evidence of underlying pre-cancerous and/or cancerous breast tissues, albeit these have typically been cluttered images with the thermal footprints of tumors and vasculature plus room-induced variations all confounded. Modern thermographic cameras are quite capable of resolving with high sensitivity discrete warm spots. The problem has been the confounding of the images due to the vasculature and operator-induced or unavoidable variations in the room and/or cooling/rewarming conditions. The ability to see surface evidence of underlying cancerous or precancerous tissue is widely thought to be at least one or both of because the tumors or diseased tissues are accompanied by increased vascularity or neoangiogenesis (increased tumor-local vasculature) as well as increased metabolism. Either or both of these cause increased local heat production, in turn causing the anomalies we are seeking to be warmer than their immediate surroundings.

The infrared spectrum is frequently described as having three major portions—the near-IR or NIR (shortest wavelengths), the mid-IR or MIR (mid-wavelengths) and the far-IR (longest wavelengths). Although the exact boundaries between these ranges apparently is not standardized across science, physics and industry, in general, the following are the values for the ranges as given by many physics and industrial journals. It will be seen that the exact boundaries between these portions of the IR spectrum are not critical to this invention, but the wavelength region being imaged is important in terms of its tissue attenuation. In other words, the dual wavelength embodiments of the invention may utilize a first non-penetrating thermal wavelength as is surface-imageable using a thermographic camera and a second penetrating wavelength. The first gives surface temperature data and the second gives subsurface (and some surface) data. The second penetrating wavelength may be delivered into the tissue as by an illumination (e.g., NIR) lamp 5C of FIG. 1 or may be excited within the tissue as by an excitation lamp 5C (excitation wavelength for particular tissue or contrast agent). The invention may utilize one, two or more cameras, illuminators or exciters to do this.
Near-Infrared: ˜1 up to 2.5 or 3.0 microns.
Mid-Infrared: ˜2.5 or 3.0 up to 14 microns.
Far Infrared: ˜14 up to 100 microns.

It will be useful to note at this point that an infrared wavelength of approximately 2.5 to 3 microns is at the bottom of the mid-IR wavelength range and the top of the near-IR wavelength range. We shall hereafter refer to this approximate wavelenght regime of 2.5-3 microns as being in the near-IR or NIR range for convenience. This IR range contains very little (but nonzero) heat-generated radiation, unlike the mid-IR or MIR range, which is dominated by heat-induced IR radiation.

Human tissue, such as breast tissue 1 of FIG. 1, typically has an infrared emission radiation peak or maximal IR emission intensity at about 10 microns wavelength—clearly in the mid-infrared, mid-IR or MIR range. This optical IR energy comes primarily from surface hotspots and not from depth. Thus, a mid-IR or MIR image of a tissue surface is a surface hotspot map and is not a direct thermal image of the hot subsurface object itself. It is an image of the heat from that object that makes it way to the tissue surface.

The present inventors realized that in the breast thermography MIR field, the heretofore unused shorter wavelengths in the near-IR or NIR (1 to 3 microns approximately) have substantial penetration in tissue, up to as much as several centimeters of depth, albeit scattering and attenuation effects take some toll on image quality from these depths. Despite that, sub-surface features can be seen in the near-IR or NIR using naturally occurring near-IR or NIR in the breast and environment, and even better using illumination by near-IR or NIR light or an illumination wavelength or excitation that excites near-IR or NIR from a near-IR or NIR contrast agent which selectively disposes itself at features of interest such as at vasculature and/or tumors.

Taken together, we realized that a first thermographic image taken at the mid-IR or MIR wavelengths (say around 8-10 microns or so) would “see” direct MIR radiation coming from the surface hotspots as for the prior art. But a second image of the same region taken at substantially the same time in the near-IR or NIR (say, at 2.5 or 3.0 microns or so) would directly “see” mostly sub-surface features with contrast originating from the feature itself, as opposed to from the heat it produces. Further, if one were to image at two or more angles of observation, one should see a shift in the NIR signature of an underlying feature relative to the MIR surface heat pattern it produces. This shift is roughly proportional to feature depth. The same kind of physical shifting can be driven by using our inventive plate/window to distort the tissue or organ.

Continuing with FIG. 1, we note item 9, which schematically represents the air or other medium that fills the space between the breast and IR camera. In the prior art mid-IR or MIR thermal imaging or thermography, this medium is typically room air. It will be seen below in at least one of our embodiments that we introduce a new material, an IR-window material, between the breast 1 and IR imager 5/5A. Our window may be used to thermally and/or physically manipulate the deformable breast in support of our inventive imaging. Our inventive window may also utilize a contact gel or liquid-particularly at the plate/breast interface.

### FIRST MAIN EMBODIMENT

The first embodiment of the invention utilizes a first nonpenetrating or MIR wavelength to image surface temperatures and a second penetrating wavelength or NIR to image subsurface features. The penetrating or NIR image information is employed in various ways to enhance a determination as to what is diseased and what is not relative to the purely thermographic prior art determination.

Some ways in which NIR or penetrating wavelength data can do this include the following:
a) using passive or excited near-IR or NIR, delineate the outlines of sub-surface features themselves from at least one point of view and more preferably from two or more points of view, such as of vasculature and/or tumors, without the confounding thermal blooming and masking effects of the mid-IR;
b) using passive or excited near-IR-, delineate any one or more of the size, shape, volume or depth of features themselves from at least one point of view and more preferably from two or more points of view, such as of vasculature and/or tumors, without the confounding thermal blooming and masking effects of the mid-IR;
c) in combination with (a) and/or (b) above, delineate or map the thermal surface MIR hotspots caused by sub-surface and surface heat-sources and sinks, themselves situated at all depths in the tissue;
d) in combination with any one or more of (a), (b) or (c) above, manipulate the tissue or object surface temperature using a source of heat or cold such as (i) blowing gas or sprayed or deposited liquids, (ii) a mid-IR radiant heater lamp, or (iii) an inventive plate/window of the second and third embodiments below; or
e) in combination with one or more of the above, utilize any manner of vasoconstriction such as cold-dipping of appendages other than the breast or as by thermal contact of a cold inventive plate/window of the second and third embodiments below.

From the above first embodiment, depending on the listed features used, one can do one or more of the following to enhance tumor identification certainty:
1) Correlate a direct near-IR or NIR image of underlying features to a mid-IR hotspot surface signature of that feature, if any.
2) Use near-IR or NIR image data to effectively subtract mid-IR heat contrast from the mid-IR image. This can be done, for example, because subsurface heat-producing (or sinking) lumens may be emitting in the near-IR and their surface hotspot components deduced and subtracted. This deduction of probable heat flow could involve modeling. It could also comprise arbitrary subtraction of a contrast amount to visually suppress the visible mid-IR contrast. Conversely, known heat producers, at least those in the form or normal vasculature or lumens, can be subtracted from the near-IR image contrast with or without the use of heat-flow modeling. Note that one would likely express both the NIR and MIR image contrast on a common contrast scale before one uses one contrast image map to modify (e.g., subtract) the other contrast image map. The remaining map can be redisplayed in any manner desired such as a “remaining or residual MIR” map. Note that such a modified map to be computed and displayed in real time as tissue temperatures change dynamically.
3) Using, for example, the above surface cooling (or heating) measures, look at the transient mid-IR surface images. For example, the rewarming rates seen at the surface will be a function of the size and depth of heat-producing tumors. A heat-production per unit volume of suspect tissue can be determined, particularly tissue known not to be a portion of a heat-producing normal vasculature. Tumors produce more heat/unit volume than healthy tissues.
4) By compressing (or suctioning) the breast and then removing the compressing means and,observing the breast at least in the mid-IR, look at the re-establishment of heat-flow as caused by reperfusion (or over-perfusion). Even more preferable is to correlate this with near-IR determined feature depths and sizes. The compressing or suctioning means in this first embodiment could be, for example, a clinician's hand or a pressure plate that can be removed such that IR imaging is then allowed. It may also be possible to do oblique loading/unloading during IR imaging-as long as the manipulator, being IR opaque, is out of the line of sight.
5) The preferred forced cooling (versus forced warming) of the breast may be done in the prior art manner or may be done using the inventive plate/window of the second and third embodiments below as discussed earlier. We include in the scope of “cooling” the cooling effect garnered by using the plate/window to squeeze the breast such that capillary blood is squeezed out. Doing this cuts off the flow of warming blood into the image field and allows the cooling plate/window, if used as a heat transfer agent, to even more effectively cool the tissue for the preferred gradual re-warming.

In summary, our first embodiment utilizes information garnered from a non-penetrating and a penetrating wavelength. Even if only one perspective view or line-of-sight is employed, it will be realized that the penetrating wavelength gives direct information about at-depth features while the non-penetrating wavelength give information about surface heat patterns which are, in large part, caused by some or all of those subsurface features. The non-penetrating data is more bioheat or function related, whereas the penetrating data is more anatomical feature shape/size/composition related.

### SECOND MAIN EMBODIMENT

The second embodiment is essentially the use of a preferably IR (MIR and/or NIR) transmissive or transparent window that can be IR-imaged through while it is also utilized to distort, shear or compress the breast. The preference is to have the window be IR transparent such that IR imaging can be performed while the tissue is distorted. However, within our inventive scope is the use of a plate/window, which is used to distort or compress the breast and is then removed for IR or other imaging without the plate present in the line-of-sight. As described earlier, the window may be flat, curved, rigid or flexible. It may be applied and/or held on the tissue by the clinician's hand, the patient's hand, by straps, clamps or actuator arms, or by suction or adhesive.

IR transmissive windows, particularly MIR-transparent windows, have been widely used in industrial applications for safety reasons, usually to isolate a worker from high voltages but to still allow MIR visualization of overheating electrical components. “Infra Red Inspection Window Materials—The Way Forward”, Nov. 9, 2005 and published on the web by GMTech Corp of Essex, England summarizes a number of available window materials offering one or both of significant near-IR (NIR) and/or mid-IR (MIR) transmissivity.

The point of this reference as employed herein is that there are IR window materials which have at least some known useful IR transparency at both mid- and near-IR wavelengths. Or to express it differently, there are window materials available for unrelated applications which pass both MIR tissue-non-penetrating light and NIR tissue-penetrating light. Some examples of the listed materials include calcium fluoride, sapphire, IR-polymer, germanium, zinc-selenide and barium fluoride. Calcium fluoride and barium-fluoride in particular might need to be shielded from moisture, as they are water-soluble over time and it may be impossible to eliminate all water or condensation from the window region. Such water film shielding could, for example, comprise a thin-film coating or an enveloping inert and/or dry gas film or blanket such as dry nitrogen or dry air. These two water-sensitive materials also need to be mechanically protected by a housing to avoid breaking them due to their comparatively fragile nature.

The distortion, shear or compression applied to the tissue by the plate/window may be initiated or sustained by the clinician's or patient's hand, by any manner of actuator arm, robot, clamp or strap or by suction or adhesive, for example. The present inventors prefer a method other than free-hand, as the reproducibility of the distortions and forces applied is otherwise more difficult to control. Most preferable will be the plate/window mounted as part of a diagnostic apparatus wherein the apparatus controls and monitors said extent and rate of distortions/shear/compression and takes image frames at controlled sampling times.

By “IR-window” we mean an element that is at least partly transmissive of at least one IR non-penetrating or penetrating wavelength utilized in the practice of the invention. Thus, it may be only near-IR transmissive, mid-IR transmissive, near- and mid-IR transmissive, or transmissive at an IR wavelength plus at a wavelength which also allows human-visual breast observation or photo-taking in a visible wavelength. As summarized by the GM Tech Corp. reference above, the window material may be rigid or flexible as for a glass window or a polymer-IR window or sheet.

More specifically, a distorting/shear or compression window of this second embodiment would favorably be used for tissue compression/shear or torsion with real-time or multi-frame through-window imaging. Essentially, the breast is forced by the IR (again, MIR, NIR or both) window to a shape, perhaps flat, and one performs thermographic mid-IR and/or or near-IR imaging through the IR transparent window from one or more lines-of-sight. During such imaging, the plate and/or the IR camera may be shifted as perhaps with regard to angle. Note that this imaging may be of the prior art mid-IR type or may be of the inventive MIR+NIR type or NIR alone. Advantages of tissue compression wherein the tissue is still IR-visible (MIR and/or NIR) through our inventive window include the following: i) we bring the underlying heat anomaly (cancer) closer to the surface, ii) we control all the optical emissivity angles, iii) we can manipulate or throttle vascular or even tumor blood flow by varying the contact pressure, iv) we can introduce an interface wetting/coating agent, if desired, which assures a known emissivity of all tissues in the field of view, and v) we can move (shear) subsurface features relative to surface features in one or both of near- or mid-IR images, thereby further isolating the structural and heat-contributions (if any) of suspect features.

Any manner of force or pressure delivery to tissue from our inventive plate is within the scope of the present invention, including application of static or dynamic forces, including vibratory, pulsatile or even ultrasonic forces or acoustic pressures. Such forces may be compressive, tensile or shearing in nature and may involve any manner of squeezing, clamping, shearing, suctioning or pulling (as using suction or adhesive).

Any manner of additional imaging modality may be combined with any of the three embodiments disclosed herein, and preferably with the plate/window-related second and third embodiments. We already mentioned mammography and ultra-sound imaging through the plates as examples of this.

For our second (and below third) embodiment, one would take, for example, a disk of the window material, say ⅛-¼ inch thick and 8 inches in diameter, and use it to apply forces to the breast. In both cases, the window will preferably allow real-time IR (MIR, NIR or both) imaging of the squeezed/sheared breast tissues, including any transient responses thereto.

We again explicitly state here that the IR window material may be rigid and flat, as it might be for pressing the breast flat using the window material, or it might be cup-shaped or conical in shape such that it fits the breast shape somewhat. It may also be flexible such that it adapts to the breast shape to some degree. The IR-polymer material mentioned in the GM Tech Corp. reference can be molded or formed to be flexible in that manner. We also note that by “pressing” or “pressure” we can mean not only pressing down on or compressing the breast, but also suctioning of the breast against a suction receptacle which may be formed using our IR transparent window material, for example. The equivalent of suction can also be practiced using a pulling member attached to the breast with an adhesive, preferably an IR-transparent adhesive. Thus, item 7 pressure or force “P” in FIG. 1 may be compression, suction, shear, torque or a combination of these as applied by inward or outward pressure, force or suction and may also be of a static, dynamic or transient nature. Preferably, such pressures or forces may be applied with the aid of our IR-transmissive window material (not shown in FIG. 1) fabricated into a convenient force-applicator shape.

The present inventors note that the plate/window IR transparent material may be mounted in a frame or non-IR window material in order to hold it and/or protect it from damage.

In FIG. 1, we noted depicted tissue coating item 11. This coating might be, for example, a sprayed alcohol per the prior art of cooling, an inventive emissivity-controlling coating such as at thin gel or cream, an inventive coating utilized between the breast 1 and an overlying IR window to minimize IR losses at the interface or to minimize emissivity errors caused by a variable interface, or even an inventive thermally conductive material that assures good thermal contact between the breast 1 and the overlying IR window. Given that, the coating may for example be any one or more of: i) applied to the breast before the exam, ii) applied to the IR window before the exam, iii) presituated on the IR window or iv) flowed into the interface of the breast 1 and overlying IR window as by pumping, gravity or capillary wetting action. In one variation, the coating is, at least in part, human sweat as produced by the breast itself.

The first embodiment, that of using a penetrating and a non-penetrating wavelength, may use a thermographic camera (non-penetrating) and a near-infrared camera (penetrating). The present inventors have utilized a thermographic camera as follows:
ThermaCAM Phoenix® from FLIR Systems
   640×512 detector, 14 bits
   Real-Time Imaging Electronics back-end
Uninterrupted Sequence Acquisition capability
Heads for each of near-IR and mid-IR ranges.
Thermographic Software: ThermaCAM Researcher™ 2.8 Professional available from FLIR Systems at www.flir.com.

For the penetrating wavelength, we have used an Hitachi CCD Model KP-F2A visible/NIR RS170 analog camera.

The images can be overlaid and compared such as by using MATLAB image processing software available from The Mathworks in Natick, Mass. or by using LabView image processing software available from National Instruments of Austin Tex. A PC, such as a Dell M65 workstation, may be used to gather and display the incoming images and, using LabView Software, one can easily compare the two different wavelength images from a given perspective or compare images at one or both wavelengths at two or more perspective views. In any event, such comparisons allow the user to determine both an apparent depth for subsurface features (two views preferred) and, from any overlying hotspot, an apparent heat-production rate possibly indicative of a tumor.

The second and third embodiments, assuming through-window imaging capability is utilized, requires an optically transmissive window. The present inventors have utilized the following materials:
1) IR-polymer material available from GMTech at www.q-m-tech.com.
2) A ceramic or glass window material such as CaF₂, ZnS, ZnSe, MgF₂ or sapphire, depending on wavelength.

Modern IR systems such as the ThermaCAM Phoenix® from FLIR can be operated in several modes. These include snapshot or frame-grabbing mode and video-mode. Because the camera is capable of a high frame rate and/or rapid sequential frame-grabs, one can view IR-contrast changes, which are transient in nature. Our analog RS-170 m combination visible/NIR camera is also capable of 30-40 frames per second.

The second embodiment calls for the window/plate to distort or deform the tissue under examination. Doing so it may be flat or curved, rigid or flexible. It may be manipulated by hand or may be manipulated by a mechanism that is part of the apparatus. In general, images or optical data from two different tissue states of compression, suction, pulling or deformation may be compared, looking for differences in behavior between surface IR signature wavelengths and sub-surface penetrating (probably NIRF or visible) wavelengths. As should be expected, deep heat-producing features, upon window shearing for example, show markedly different image lateral motions between the two states of deformation, with the thermal tissue surface image following the shearing window interface motion and the deeper penetrating contrast image not moving nearly as much. The relative motion is proportional to feature depth. Note that immediately upon tissue shearing, the surface hotspot rotates with the window, whereas the underlying penetrated image moves less. After several seconds, the surface hot spot reappears, overlying the more stationary penetrating image. This is simply because the underlying heat-producing tumor creates a new hot spot in its new overlying tissue. Some amount of window rigidity is preferable if the tissue is to be forcefully deformed as described.

The third embodiment requires the plate/window to act as a means of heat transfer such that underlying tissue can be heated or cooled. To do this, the window one or more of (a) is itself pre-cooled or pre-warmed before tissue application, (b) has integrated heat-exchanger means in it or on its surface, and (c) acts as part of a container through which tissue-contacting heat-exchange fluid or gel is pumped or passed. If the window-underlying heat exchange fluid is used (approach (c)) the fluid needs to be optically transparent to at least one wavelength of interest.

Given the need to manipulate thermal energy, it is desirable to use a plate with a significant heat capacity and plate pre-warming or pre-cooling (before tissue contact) such that one can completely avoid plate mounted heating or cooling means. However, the present inventors have also utilized optical windows that have arrayed thin-film heaters such as indium tin-oxide transparent heaters. Typically, the breast may be cooled, such as by a pre-cooled or self-cooled plate and the re-warming of the breast can be observed in the typical thermographic exam fashion, except here we have far more control over the rate and uniformity of the cooling and/or re-warming. Note also that tissue contacting a flat plate/window is roughly normal to any thermal IR camera, thereby assuring minimization of the variation of optical emissivity due to angle of observation.

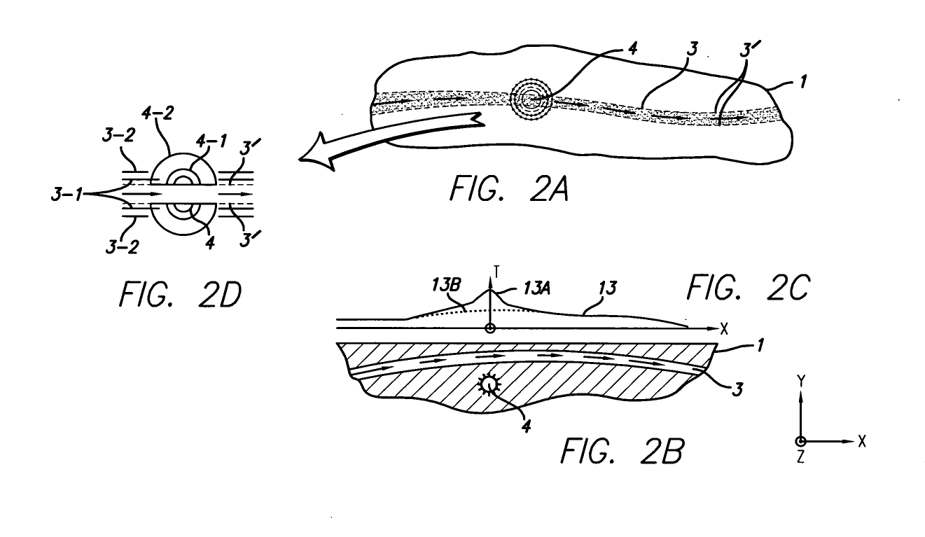

Turning now to FIGS. 2A-2D, we see a top plan view (FIG. 2A) and front sectional view (FIG. 2B) of a breast tissue portion 1 containing a heat-producing vascular lumen 3 underneath of which resides a heat-producing tumor 4. In FIG. 2A, it will be noted that the physical edges of the lumen 3 are designated as 3′. By “physical edge” we mean the physical material boundaries. Note also the coordinate system on the right of FIGS. 2A-2D, wherein we have the X-Y plane in the drawing and the Z-axis coming outwards toward the reader. We indicate with arrows inside of lumen 3 a flow of blood rightwards.

As shown in FIG. 2A, the tumor 4 substantially underlies the lumen 3. Now if we were to thermographically image this top tissue surface in the prior art thermographic surface mid-IR, what one would see is superimposed hotspots from both the lumen and the tumor. One would also probably see the hottest spot over the combined lumen 3 and tumor 4, as that is where the most heat is being leaked out. FIG. 2C depicts a temperature profile taken along the length of the substantially straight lumen 3 and passing through tumor 4. The temperature profile 13 is shown as having a peak temperature at the aforementioned coincident tumor/lumen overlap site. Phantom temperature line 13B depicts the temperature map we would have seen had the tumor not been present. FIG. 2B depicts the tissue of FIG. 2A in section. FIG. 2D is an enlargement of the coincident tumor/lumen region of the top view depicted in FIG. 2A. Again, tumor 4 is seen substantially underlying lumen 3 having lumen edges 3′. However, in the enlargement, we also see thermographic mid-IR temperature gradients around the tumor indicated as 4-1 and 4-2 as well as temperature gradients around the lumen 3 indicated as 3-1 and 3-2.

It will be appreciated at this point that if one utilizes mid-IR thermographic (heat) imaging only seen at tissue surfaces, one will see nothing but the hotspots and their gradients at the tissue surface and will not see the actual physical edges of the tumor 4 nor the lumen 3. However, if we were to view the tissue surface in near-IR wherein we have some penetration ability, we will see, instead, outlines of the physical edges of the tumor and lumen.

If one were to assign a common contrast scale to both of the mid-IR and near-IR images and express their contrast on that common scale, one could use one type of data to modify the other. For example, if we express both types of contrast on a common scale, then subtract the near-IR contrast data from the mid-IR contrast data, and then re-express the result on the mid-IR contrast scale, one would essentially suppress mid-IR contrast whereat there appears near-IR contrast. What one would get is shown in FIG. 2D, namely, a contrast image of the tumor portions not underlying the lumen 3. This appears as a circle with a chunk missing from its mid-portion as shown.

The principle being taught here is that one can modify one data set (the mid-IR data set in this example) with another data set (the near-IR dataset in this example). The art of image manipulation is long and deep, originating in the intelligence and technology communities. What we did above is to use NIR data to identify elongated lumens. We then said, knowing that the elongated lumens also have a heat signature, that we could use the NIR data to subtract out an assumed thermal effect of the lumens. In fact, the lumens, being typically closer to the surface, are even more visible in the NIR so their corresponding estimated thermal IR contrast can effectively be subtracted or suppressed from any remaining thermal contrast. Note that this subtraction or suppression accomplished, more or less, what vasoconstriction accomplishes. We note that before one type of wavelength data is used to modify another, one can take multi-perspective views.

So, using the first embodiment, one can improve an optical image using a second different wavelength optical image as we depict in FIGS. 2A-2D.

It will be appreciated, for example, that if one can see multiple structures in the near-IR but they do not have corresponding hotspots, then they are probably not heat-producing cancer. Of course, they may also be an infection, but prior thermographers are aware of the symptoms of such conditions and would have the patient treated for that instead. We note again in FIG. 2C that the temperature plot 13 would follow dotted lines 13B if the tumor were not present.

One may utilize heat-flow modeling in combination with our inventive image manipulation of embodiment 1 or of the later embodiments 2 and 3 below. As a specific example, we could gather mid-IR surface images and near-IR penetrating images of the same region, say the region shown in FIGS. 2A-2B. From these two images, one can recognize that one has a “point” heat source (the tumor) superimposed on a linear heat source (the lumen). From having near-IR images at an angle or at multiple angles, it can be ascertained in the near-IR that the tumor underlies the lumen, if that is not already obvious to the clinician. Now one can have software compute and subtract from the mid-IR image all mid-IR heat patterns that correspond to lumens. Since the heat output of the lumen itself is observable in regions away from the tumor, one can easily “fill-in” or predict what heat pattern would be present in the tumor region due to the lumen if the tumor had not been there. Thus, after subtracting the lumen heat, one is left with the tumor residual heat as a localized hotspot without a lumen-related overlying hotspot running through it. Since we also know the depth and size of the suspect-tumor from the near-IR views, we have all of the following information: a) tumor size, b) tumor depth, and c) tumor surface heat-signature in the mid-IR. Given these, one can model what heat output the tumor must have per unit volume of tumor tissue in order to create that specific surface hotspot. Thus, one can obtain a milliwatts/cm³ heat output of suspect tissue—a number that surely is going to correlate with anomalous heat-output.

Note that in the above exercise, we applied some assumptions and some modeling. In FIG. 2D discussed earlier, we took the easiest and basic approach, namely, just subtract one image from the other after expressing them on a common contrast scale and then reconvert the result to the mid-IR scale. That indeed gets rid of the lumen mid-IR contrast, but it also gets rid of coincident superimposed mid-IR contrast due to the underlying tumor. So in that crude approach, the round tumor looks like a circle of heat with the middle chopped out, as shown in FIG. 2D.

The point here is not to claim specific algorithms for image conditioning, as there are hundreds of possibilities, many of them offering useful increases in signal-to-noise of tumor identification (or even of lumen identification). What we are really claiming here is the creation of new and additional information that can be used in a multitude of algorithms to offer the needed signal-to-noise (hereafter called S/N) improvement. Note that the new information in the above examples not only works with the old information (mid-IR info), but it also works alone in reporting depths and sizes of structures. So it is more correct to call it new information useful both to improve the old data as well as to provide different new data.

### THIRD MAIN EMBODIMENT

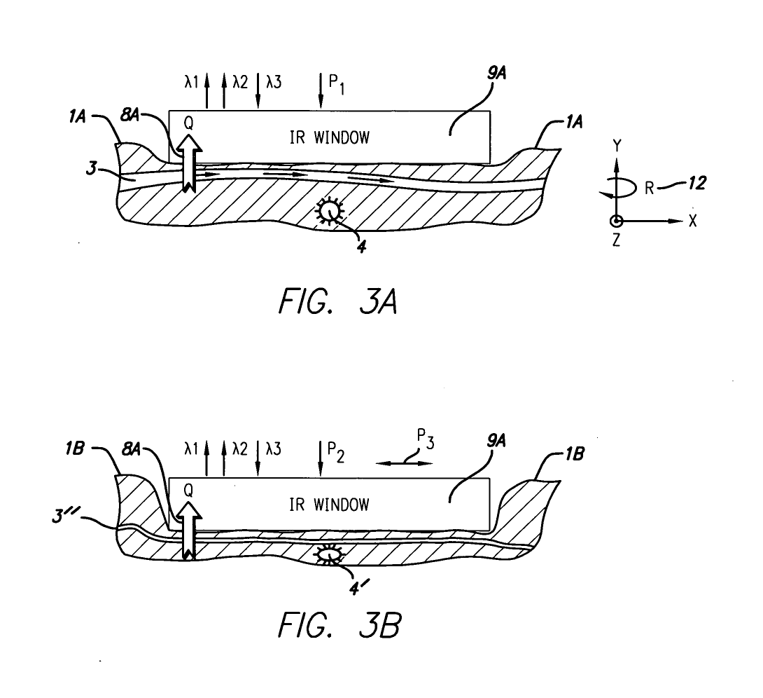

Moving now to FIGS. 3A-3B, we will describe the use of the second (deforming) plate/window embodiment and the third (heat-manipulating) plate/window embodiment of the invention. The plate/window is a means to thermally and/or physically manipulate tissues while preferably being able to simultaneously observe them at one or more optical wavelengths. The second and third embodiments are not limited to using a penetrating and a non-penetrating wavelength in combination like the first embodiment. Embodiments 2 and 3 may use only one wavelength or may use two or more wavelengths. Embodiments 2 and 3 preferably utilize two or more different states of tissue-deformation and/or tissue-temperature in combination with one (or more) viewing or detection wavelengths.

A rigid or semi-rigid optical window having a significant thermal capacity, such as our example window materials, can be used to deform tissue and/or inject/remove heat from tissues into or from the window's own thermal mass. Because the IR window can have a significant thermal capacity (unlike the air or blown air 9 of FIG. 1) and can be optically transparent to surface mid-IR, one can view short-time thermal transients of any magnitude through the window.

It will also now be apparent that the IR window or optical window of embodiment 2 can be used to squeeze tissues in a manner such that the distances to tumors change and perfusion and blood flow in lumens and tumors change. Mechanically inclined readers will know that such effects fall off in magnitude with depth as well. Thus, we explicitly claim forced modulation of tissue or lumen (or even tumor) perfusion or flow by applying window/plate tissue deformations. Note that this is a mechanical effect as opposed to the nervous-system reaction involved in vasoconstriction. The same applies to apparent dimensional changes upon such squeezing or deformation—such deformations give information about diameter, compliance and depth.

FIGS. 3A and 3B are each sectional front views of a tissue region similar to that shown in FIG. 2B. The tissues under investigation are shown as more compressed or deformed in FIG. 3B than in FIG. 3A. In FIG. 3A, an IR window 9A is lightly contacting the minimally deformed tissue 1A. In FIG. 3B, we show the same IR window 9A after having applied a larger, more significant pressure load to the now increasingly deformed tissue 1B. The light initial load is indicated by force or pressure P₁ whereas the subsequent larger significant deformation load is depicted as load or pressure P₂. We explicitly note in FIG. 3B an alternative or additional load P₃ shown as a shearing load. This shearing load is discussed in further detail below.

Note that when switching from the light load P₁ to the heavier load P₂, the lumen 3 becomes compressed if not squeezed substantially shut to flow as depicted by 3″. Note also that the same higher load P₂ has squeezed the tumor 4 to a deformed state 4′. Such deformations alter blood flow and therefore heat output as well as alter apparent lateral dimension as viewed in, for example, penetrating near-IR.

We show in both FIGS. 3A and 3B a heat-flow Q of the third embodiment which is typically a cooling of the tissue by a cooler IR-window 9A followed by tissue re-warming. Useful vasoconstriction may also take place due to the application of the cooling plate. The scope of the invention includes any heat-flow inwards and/or outwards as delivered by thermal conduction (shown) or as delivered, for example, by a radiant or electromagnetic energy source (not shown). As an example, the IR-window could be pre-cooled, it could have an integrated cooler, or it could have heating radiant IR-lamp energy directed through it, or even microwave energy.

We show in FIGS. 3A-3B three wavelengths of at least IR passing into or out of the IR window and tissue. As we previously mentioned, out-going (from tissue 1A, 1 B) λ₁ and λ₂ wavelengths could be, for example, mid-IR and near-IR wavelengths. In-going (to tissue 1A, 1B) λ₃ wavelength could be, for example, the prior discussed near-IR illumination or excitation.

We emphasize that the invention may utilize other wavelengths such as that of a human-visible video camera, an X-ray machine or an MRI (RF excitations), for example.

In our Figures, we have shown a single IR-capable camera and a single IR window (in the window embodiments), both generally operated in a head-on orientation into or onto the tissue. We now emphasize that the invention is not limed to head-on imaging or tissue manipulation. As an example, one might utilize a tissue-clamping arrangement similar to mammography (perhaps combined with a mammography capability) wherein a window is provided on one or both clamped faces of the tissue. Furthermore, the invention includes front-lighting (depicted herein) as well as back-lighting and side-lighting. Back-lighting, for example, may be done in the mammography arrangement wherein the near-IR light source is on one face (the back) and the near-IR imager is on the other face (the front).

Also included in the scope is the use of invasive or minimally invasive surgical tools to, for example, biopsy tissue or re-sect tumor tissues. Such surgical measures may be accompanied by the use of another imaging modality or not. Near-IR imaging of a biopsy needle may be done using the teachings of the present invention.

One may also co-integrate other modalities into our inventive IR window. As an example, the window may contain one or more holes in it through which ultra-sound imaging or minimally invasive surgery is performed. The window may have IR-visible or human visible markers or scales on it. The window may have spatial encoders such that the computation means knows exactly where it is relative to a reference point or points.

Continuing with FIGS. 3A-3B, we wish to further discuss shearing force or load P₃ shown in FIG. 3B. A shearing load, which can be applied by, for example, translation (P₃) or rotation (R 12) of the IR window 9A, has the ability to differentially displace shallow features relative to deeper features. This phenomenon can be very useful to sort out confounding overlying image contrast, whether it be in the mid-IR or near-IR. In addition, tumors may demonstrate a unique deformation behavior different that that of surrounding healthy tissues.

Thus, we have two ways to get three dimensional information, a) one or more near-IR images which can see beneath tissue, and b) tissue deformation while under one or both of near-IR or mid-IR observation. Note that if tissues are sheared in a way that moves an underlying tumor out from under an overlying lumen. For example, then both the near-IR image and the mid-IR image will see that because both the actual tumor moves as well as, after a time period, its surface hotspot contribution.

So using our inventive optical or preferably IR-window/plate 9A, we can deformably manipulate tissues under study in a multitude of thermal and/or physical ways, including ways that in turn affect blood flow and perfusion. The present inventors have provided several new variations that may be tried to sort out exactly what tissues are present and how they act physically and thermally.

The present inventors anticipate utilizing the invention in the form of an apparatus preferably including an area-wise IR camera(s) or imager(s) such as the taught FLIR unit. However, IR-imaging inclined readers will realize that one may also utilize, rather than M×N two dimensional area-wise image-capture detectors, single row detectors with N elements that are scanned along the third axis. In an extreme case, one could utilize a single element (N=1) detector which measures the IR at a single point and that single point is scanned or otherwise directed to or across potential tumor sites in a raster or vector pattern.

Per the prior art, we also include in the scope of the invention the stressing of the patient's physiology, as by exercise or drug-induced stress. Finally, we also include the concept, now apparent, of having a combined apparatus that does both the inventive surface thermography or other at-depth optical, imaging as well as another different form of imaging such as mammography, simultaneously or sequentially. Doing this would allow for a nicely registered set of images that can be computer- or radiologist-compared in a manner providing synergistic and reinforcing diagnosis.

The present invention may be utilized non-invasively or invasively. In an invasive situation, one might observe a bodily organ or tissue across an air (or insufflation CO₂) gap, for example, or one may observe the same organ through our contacting optical window. Note that using an IR optical window, one could physically displace or exclude blood from the IR line of sight if desired, thus making possible under-blood (or other bodily fluid) tools such as in gastroscopic, laparoscopic, colonoscope, bronchoscope or endoscopic form factors.

The invention herein, particularly when using the plate/window second and third embodiments, is expected to minimize the undesirable effects of breezes in the examination room. Within our inventive scope for use with either second or third inventive embodiment is the use of additional thermal insulation means or clothing that assures that the breast being examined and/or the patient is not being affected by uncontrolled heat inputs and outputs such as by breezes or sunlight.

We note that there will be a lower loading force P₁ sufficient to assure intimate thermal and/or optical contact of the IR-window to the target tissue 1A. In order to significantly deform tissues purposely, a higher load P₂ and/or P₃ will likely be utilized.

Also within the scope of the invention are non-solid optical or IR window materials, such as those formed from gels or liquids, including disposable windows or window materials.

It will also be appreciated that if near-IR radiation from tissues is excited, as by a laser, for example, then the window may also be transparent to the excitation wavelength. The same applies to direct illumination with a near-IR source or mid-IR source; we have both in-going and out-coming near-IR energy.

There are several causes of heat-anomalies in or on living tissue beyond cancer, such as infections and a host of metabolic diseases. The scope of the present invention includes any such anomaly in or on any living tissue. By “tissue” we mean any living tissue or matter in a human or animal. Such a definition includes skin, organs, body fluids and bone structures.

It will now be obvious that any of the three embodiments may be used alone or together. Using them together offers several simultaneous new ways to image the reaction of suspect tissues at any of different wavelengths, different mechanical loads, or different thermal states. Embodiments 2 and 3 are easily combined since a contacting plate or window can easily both transfer heat and apply forces. By combining the embodiments we mean sequential or simultaneous implementations.

What is claimed is:

1. An apparatus for the optical detection of abnormality or disease in a human tissue or anatomy portion, the apparatus utilizing two optical wavelengths, a first non-penetrating wavelength and a second penetrating wavelength, wherein the first wavelength is a thermal IR wavelength emitted primarily from the surface providing primarily surface temperature information relating at least partly to thermally-coupled underlying sub-surface features and the second wavelength is a tissue penetrating IR wavelength providing direct subsurface contrast information, the two types of data being correlated or compared such that two different signatures of the same abnormality reinforce the certainty that what is being seen is a subsurface heat-producing abnormality, the apparatus comprising:
a detector or imaging camera for detecting or imaging the first thermal related non-penetrating surface-emitted wavelength;
a source of or exciter of the subsurface second penetrating wavelength which give contrast information;
a detector or imaging camera for detecting or imaging the emitted, reflected or through-transmitted second penetrating wavelength; and
a means to compare data from both wavelengths with regards to at least one point or region of suspected or potential abnormality, a correlation of the two types of data capable of indicating a sub-surface feature that also emits thermal energy and may be a tumor, infection or other heat-producing abnormality.

2. The apparatus of claim 1 wherein the room lighting provides or excites most or all of the second penetrating optical wavelength.

3. The apparatus of claim 1 wherein a laser, flash-lamp, LED or other optical exciter is directed onto or into tissue such that said tissue then produces the second penetrating wavelength.

4. The apparatus of claim 1 wherein any of:
a) two separate detectors or cameras are utilized, sequentially or simultaneously, to gather optical data;
b) at least one detector or camera is or is also capable of detecting or imaging in a human-visible wavelength;
c) at least one detector or camera utilizes a CCD or CMOS imaging chip;
d) an optical contrast agent is utilized;
e) a first (or second) wavelength causes emission of the second (or first) wavelength);
f) image clutter due to veins or arteries is reduced as by pattern-recognition of lumens and feature subtraction or suppression and/or by vasoconstriction;
g) an image or image point in one wavelength is modified using an image or image point in the second wavelength with the purpose to reduce image clutter or noisiness; or
h) tissue or anatomy is imaged as it cools, re-cools, warms or re-warms.

5. An optical window apparatus that is placed in contact with anatomy suspected of being diseased or abnormal, the window causing at least some conformation of the anatomy to the window shape during contact, the window being at least partly transparent to an optical wavelength, said wavelength being detected or imaged from outside the window and through said window, said anatomy being deformable by said contacting window comprising:
a) an optical window member through which at least one wavelength of optical energy useful for inspecting or imaging tissue can pass outwardly;
b) the optical window brought into contact with the suspect tissue, thereby conforming at least some such tissue to at least a portion of the window, said window-contacting being manual or being assisted by the apparatus;
c) the tissue observable during window contact using the at least one wavelength, which can pass from the tissue outwardly through the window for at least one of aided or unaided observation or measurement;
d) the outwardly passing optical wavelength being one or more of a: i) a thermal IR wavelength emitted from the tissue surface region, ii) a near infrared tissue-penetrating wavelength emitted, reflected or attenuated by subsurface anatomical features, or iii) a visible wavelength emitted, reflected or attenuated by subsurface anatomical features;
e) the tissue observable at at least one state of deformation at at least one said wavelength;
f) the at least one tissue deformation state being or including one or more of 1) squeezing by, adherence to or a suctioning to the window, 2) lateral translation or shearing by the window, 3) rotational or torsional shearing by the window, or 4) any tilting or dynamic motion of the window causing tissue deformation; and
g) at least one said deformed tissue image providing data, optionally in combination with another one or more deformed images or an un-deformed image before the window is contacted, revealing the telltale different image behavior of features at different depths or of features and their corresponding surface thermal signatures.

6. The apparatus of claim 5 wherein two images at two different states of tissue deformation are compared, said comparison revealing at least some information about 1) the relative depth of features, 2) the relative depth of features having surface thermal signatures, 3) the depth of any feature, 4) a difference in imaging relating to the blood being substantially squeezed out, or 5) a difference in imaging relating to lumens and/or tumors being flattened or collapsed.

7. The apparatus of claim 5 wherein at least one optical wavelength emitted outwardly through the window is one of: a) a tissue-surface emitted thermal IR wavelength, b) a wavelength which can penetrate tissue and therefore is passed from within said tissue out of the tissue, c) a tissue penetrating infrared or visible wavelength, d) a wavelength which is a constituent of a reflected illumination directed through or under the window, or e) a wavelength which is excited by an illumination excitation directed through or under the window.

8. The apparatus of claim 5 wherein the window material chosen is an infrared or visible window material.

9. A heat exchanging plate or window apparatus used to thermally manipulate or thermally control anatomical tissues being examined for disease or abnormality comprising:
a) an optically opaque plate or optically transmissive window member which is juxtaposed to tissue in conforming direct thermal contact or at a standoff gap;
b) any standoff gap being filled with a thermally conductive flowable or conformable medium such as a thermally conductive liquid or gel;
c) the anatomical tissue under study having its thermal state manipulated by heat transferred into or out of the tissue from or to the overlying gapped or contacting plate/window and/or any heat-exchange medium flowed through or placed into any such gap;
d) the tissue being optically observable at at least one non-penetrating or penetrating wavelength either through said window or window/medium while it is in place or being observable after an opaque heat-exchange plate is removed; and
e) said thermal manipulation serving to provide or enhance an optical contrast of the tissue.

10. The apparatus of claim 9 wherein a tissue portion is cooled for observation during said cooled state or during a re-warming.

11. The apparatus of claim 9 wherein a tissue portion is warmed for observation during said warmed state or during a re-cooling state.

12. The apparatus of claim 9 wherein some tissue is thermally vasoconstricted.

13. The apparatus of claim 9 wherein the plate/window any of: a) has an internal or integrated heater or cooler mechanism, b) is thermally coupled to a flowed coolant or heating medium, c) serves to contain a thermally conductive medium between it and an underlying tissue portion, d) contains a temperature measurement device, e) is preheated or pre-cooled in a separate environment before tissue placement, f) has thermal infrared transmissivity, g) has near infrared transmissivity, h) has visible transmissivity, or i) contains optical illumination or excitation means or acts as an ingoing window for such means.

14. An apparatus for optically examining human tissues for disease or abnormality utilizing, simultaneously or in sequence, any two or more of the following members:
a) an optical window through which a tissue penetrating and a tissue non-penetrating optical wavelength each can be passed through said window in at least one direction;
b) an optical window which is placed in contact with anatomy suspected of being diseased or abnormal, the window causing at least some conformation of the anatomy to the window shape during contact, the window being at least partly transparent to an optical wavelength, said wavelength being detected or imaged from outside the window and through said window, said anatomy being deformable by said contacting window; and
c) a heat exchanging plate or window used to thermally manipulate or thermally control anatomical tissues being examined for disease or abnormality, said plate or window directly thermally contacting the tissue or being thermally coupled to tissue via a standoff gap filled with a thermally conductive flowable or deformable medium,
wherein at least one of the two or three members passes at least one optically detectable or imagable tissue-penetrating or non-penetrating wavelength outward to an observing detector or camera.

15. The apparatus of claim 14 wherein tissue is warmed or heated by: i) thermal infrared radiation directed onto or into tissue through or from a window, or ii) thermally-conducted heat from a heat-exchange plate/window.
